(19) United States
(12) Patent Application Publication
(10) Pub. No.: US 2020/0315853 A1
(43) Pub. Date: Oct. 8, 2020

(54) WOUND DRESSING FOR THE HARVESTING OF SUPERFICIAL EPIDERMAL GRAFTS

(71) Applicant: KCI Licensing, Inc., San Antonio, TX (US)

(72) Inventor: Alexander WAITE, San Antonio, TX (US)

(21) Appl. No.: 16/955,759

(22) PCT Filed: Dec. 14, 2018
(86) PCT No.: PCT/US18/65815

§ 371 Date: Jun. 18, 2020

Related U.S. Application Data
(60) Provisional application No. 62/608,280, filed on Dec. 20, 2017.

(51) Int. Cl. A61F 13/00; A61B 17/322; A61L 15/40; A61L 15/22

(52) U.S. Cl. CPC A61F 13/00; A61B 17/322; A61L 15/40; A61B 2017/00969; A61F 2013/00425; A61F 2013/00859; A61L 15/225

(57) ABSTRACT

In some aspects, the present invention provides an active dressing for transfer of superficial epidermal biopsies to a wound site, where the dressing can enhance keratinocyte out growth from the biopsies at the wound site and improve the rate of re-epithelization. In some embodiments, the dressing can include a perforated dehydrated amniotic membrane sheet that is coupled to a backing sheet having suitable adhesive properties. The perforations can have appropriate dimensions and distributions for capturing and transferring the skin biopsies. Without being limited to any particular theory, the amniotic membrane sheet can enhance the re-epithelization of the wound site to which the biopsies are transferred, e.g., by supplying signaling proteins and extra-cellular matrix (ECM) components.

### BACKGROUND

The present invention relates generally to dressings for transferring skin grafts, e.g., epidermal skin grafts, from a donor site to a recipient site, and more specifically to dressings that employ amniotic material to enhance the re-epithelization of a recipient site.

Skin grafting is a surgical procedure in which a section of skin is removed from one area of a person's body (autograft), removed from another human source (allograft), or removed from another animal (xenograft), and transplanted to a recipient site of a patient, such as a wound site. Chronic wounds are often observed in elderly patients and/or in patients with severe comorbidities. A common feature of different types of chronic wounds is impaired re-epithelization. Autologous epidermal grafting provides a solution to wound closure by transferring the patient's own epidermal cells to the wound site.

A device marketed by the assignee of the present application under the trade designation Cellutome® can be used to create and harvest such epidermal grafts in an automated and precise manner. The Cellutome® system allows harvesting of superficial autologous skin grafts/biopsies in a minimally invasive manner. The application of autologous skin grafts has been shown to be effective in the treatment of superficial, granulated wounds that are free of necrotic tissue and infection. The system generates a series of microdomes/biopsies from healthy skin, which are harvested and collected using an adhesive film. The resulting superficial biopsies have been shown to have a high level of cell viability. In addition, cellular outgrowth has been demonstrated in vitro from biopsies indicating ability of cells to migrate and proliferate. Currently biopsy transfer is predominantly conducted using either an adhesive film or a silicone contact layer dressing.

There is a need for improved dressings that allow facile capture of skin grafts, e.g., epidermal skin grafts and also enhance re-epithelization of a recipient site to which the skin grafts are transferred.

### SUMMARY

In one aspect, a dressing for transferring skin grafts from a donor site to a recipient site is disclosed, which comprises a perforated amniotic membrane sheet having a skin-graft contacting surface and an opposed surface, and a backing sheet having a surface coupled to said opposed surface of the amniotic membrane sheet. In some embodiments, the perforated amniotic membrane sheet can include one or more perforations configured for harvesting one or more epidermal skin grafts, e.g., via tacky portions of the back sheet that are in register with the perforations.

By way of example, in some embodiments, the perforations can have a width in a range of about 2 mm to about 10 mm. Further, in some embodiments, the perforated amniotic membrane sheet has a thickness in a range of about 5 μm (micrometers) to about 100 μm.

In some embodiments, the surface of the backing sheet that is coupled to the amniotic membrane sheet can be coated at least partially with an adhesive so as to provide one or more adhesive sites in substantial register with said one or more perforations so as to facilitate harvesting of epidermal skin grafts via said perforations.

In some embodiments, the perforations are distributed across the amniotic membrane sheet as a regular array (e.g., a regular two-dimensional array). The amniotic membrane sheet can include any of amnion and/or chorion.

The backing sheet can be formed of a plurality of suitable materials. By way of example, in some embodiments, the backing sheet is formed of a polymeric material. Some examples of suitable polymeric materials include, without limitation, polyurethane, polyolefins such as polyethylene, polypropylene, and cyclic olefin copolymers, polyamides, polyesters and polyethers, a co-polyester or a copolymer thereof, or a blend thereof.

In some embodiments, the backing sheet can have a thickness, e.g., in a range of about 15 μm to about 100 μm.

In some embodiments, the adhesive can be any of silicone, acrylic, polyurethane or hydrogel-based adhesives.

The dressing can have any size and aspect ratio suitable for an intended application. For example, the dressing can be sized so that it can fit within a chamber of a Cellutome® device to capture a plurality of cut epidermal blisters.

In a related aspect, a dressing for transferring skin grafts from a donor site to a recipient site is disclosed, which includes a backing sheet having a surface, which includes one or more adhesive sites, and one or more amniotic membrane sites distributed across said surface of the backing sheet such that said one or more adhesive sites remain exposed for harvesting one or more skin grafts.

In some embodiments of the above dressing, the amniotic membrane sites include one or more strips including amniotic material. By way of example, the amniotic membrane sites can include a plurality of strips distributed across the surface of the backing sheet so as to form a crisscross pattern, where the strips include an amniotic material. In some such embodiments, the adhesive sites can be distributed across said surface of the backing sheet such that any two adhesive sites are separated by at least a portion of one of said strips.

In the above embodiment, the adhesive sites can have a variety of different shapes. By way of example, in some implementations, the adhesive sites can be substantially circular. In other embodiments, the adhesive sites can have a polygonal shape.

In some embodiments of the above dressing, the amniotic membrane sites can be in the form of a plurality of dots that are distributed across the surface of the backing sheet. By way of example, the dots can have a diameter in a range of about 1 mm to about 10 mm.

In some embodiments, the adhesive sites can be formed via a coating of an adhesive material, such as those discussed above. Further, the backing sheet can be formed of a suitable polymeric material, such as those discussed above. The backing sheet can have a thickness in a range of about 15 μm to about 100 μm, though other thicknesses can also be employed.

In a related aspect, a dressing for transferring skin grafts from a donor site to a recipient site is disclosed, which includes a backing sheet having a surface providing one or more adhesive sites, and a powder comprising an amniotic membrane material, said powder being distributed across at least a portion of said surface of the backing sheet such that said one or more adhesive sites remain exposed for harvesting one or more skin grafts.

In some embodiments, the powder can include a plurality of particles having a size in a range of about 1 micron to about 300 microns. In some embodiments, the adhesive sites are distributed as a regular array across said surface of the backing sheet. The adhesive sites can have a variety of different shapes, such as circular, polygonal, etc.

In some embodiments, the powder can include collagen. In some such embodiments, the powder can include collagen and oxidized regenerated cellulose (ORC).

In a related aspect, a dressing for transferring skin grafts from a donor site to a recipient site is disclosed, which comprises an adhesive biopolymer film having a surface adapted for harvesting skin grafts, and a powder comprising an amniotic membrane material, said powder distributed across at least a portion of said surface of said adhesive biopolymer film. The amniotic membrane material can be amnion and/or chorion. In some embodiments, the adhesive biopolymer film comprises collagen. In some such embodiments, the adhesive biopolymer film can include collagen and ORC. In some embodiments, the powder can include a plurality of particles having a size in a range of, e.g., about 1 micron to about 300 microns.

Further understanding of the invention can be obtained by reference to the following detailed description in conjunction with the associated drawings, which are described briefly below.

### BRIEF DESCRIPTION OF THE DRAWINGS

FIG. 1A is top view of a dressing according to an embodiment,

FIG. 1B is a cross-sectional view of the dressing depicted in FIG. 1A,

FIG. 2 is a top schematic view of a dressing according to another embodiment,

FIG. 3 is a top schematic view of a dressing according to another embodiment,

FIG. 4A is a top schematic view of a dressing according to another embodiment,

FIG. 4B is a schematic cross-sectional view of the dressing depicted in FIG. 4A,

FIG. 5 is a top schematic view of a dressing according to another embodiment,

FIG. 6 is a top schematic view of a dressing according to another embodiment,

FIG. 7 is a top schematic view of a dressing according to another embodiment,

FIG. 8 is a top schematic view of a dressing according to another embodiment,

FIG. 9A is a schematic view of a device for generating a plurality of epidermal skin blisters, and

FIG. 9B shows a multi-plate cutter mechanism employed in the device of FIG. 8A.

### DETAILED DESCRIPTION

In some aspects, the present invention provides an active dressing for transfer of superficial epidermal biopsies to a wound site, where the dressing can enhance keratinocyte out growth from the biopsies at the wound site and improve the rate of re-epithelization. In some embodiments, the dressing can include a perforated dehydrated amniotic membrane sheet that is coupled to a backing sheet having suitable adhesive properties. The perforations can have appropriate dimensions and distributions for capturing and transferring the skin biopsies. Without being limited to any particular theory, the amniotic membrane sheet can enhance the re-epithelization of the wound site to which the biopsies are transferred, e.g., by supplying signaling proteins and extra-cellular matrix (ECM) components.

Various terms are used herein according to their general understanding in the art. The term “amniotic membrane sheet” as used herein refers to a sheet that contains amniotic membrane material, such as, amnion and/or chorion. The term “about” as used herein to modify a numerical value denotes a variation of at most 5%. The term “substantial,” e.g., as in the phrase “in substantial register,” denotes a deviation, if any, of at most 5% relative to a perfect state or condition.

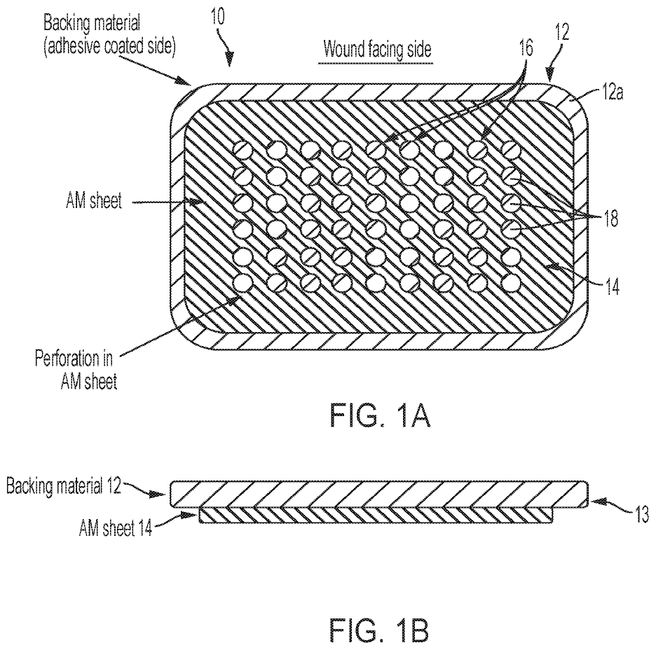

FIGS. 1A and 1B schematically depict a dressing 10 that is configured to receive one or more epidermal skin grafts and transfer the skin grafts to a recipient site, e.g., a wound site. The dressing 10 includes a backing sheet 12 that includes a wound-facing surface 12a to which a suitable adhesive coating 13 has been applied.

In many embodiments, the backing sheet 12 can be a breathable protective film. In some embodiments, the backing sheet can include an absorbent component, such as foam. By way of example, the backing sheet 12 can be formed of a suitable polymeric material. Some examples of suitable polymeric materials can include, without limitation, polyurethane, polyolefins such as polyethylene, polypropylene, and cyclic olefin copolymers, polyamides, polyesters and polyethers, a co-polyester or a copolymer thereof, or a blend thereof.

The adhesive film 13 can be formed of any suitable adhesive. Some examples of suitable adhesives can include, for example, silicone, acrylic, polyurethane or hydrogel based adhesives.

The dressing 10 further includes an amniotic membrane sheet 14 that is disposed over and adhesively coupled to the surface 12a of the backing sheet 10. In this embodiment, the amniotic membrane sheet 14 includes a plurality of perforations 16 that expose a plurality of adhesive portions 18 on the underlying backing sheet 12. These adhesive portions advantageously facilitate the capture of skin grafts and their transfer to a recipient site.

In many embodiments, the spatial arrangement and dimensions of perforations 16 formed in the amniotic membrane sheet 14 can be configured to allow facile capture of epidermal skin grafts generated by an epidermal biopsy system of interest. By way of example, in this embodiment, the perforations 16 are distributed as a regular array. In some embodiments in which the dressing 10 is intended for capturing a regular array of epidermal blisters the perforations 16 can be arranged to be substantially in register with the epidermal skin grafts so that the dressing can readily capture the skin grafts for transfer to a recipient site. By way of example and as discussed in more detail below, in some embodiments, epidermal blisters can be formed via application of a negative pressure to a housing to raise a plurality of epidermal blisters into a plurality of openings of a plate attached to a distal end of the housing,

In some embodiments, the amniotic membrane sheet 14 can have, for example, a thickness in a range of about 5 to about 80 microns. Further, in some embodiments, the perforations can have a maximum lateral dimension, e.g., a diameter in the case of substantially circular perforations, in a range of about 2 mm to about 10 mm. Although in this embodiment the perforations are depicted as substantially circular, in other embodiments, they can have other shapes, e.g., a polygonal shape.

Without being limited to any particular theory, when the dressing 10 is placed on a recipient site, e.g., a wound site, to transfer the captured skin grafts to the recipient site, the amniotic membrane sheet can advantageously modulate the wound environment, e.g., by supplying growth factors, anti-inflammatory cytokines and extracellular matrix (ECM) components. This can in turn enhance the process of re-epithelization through modulation of keratinocyte migration and proliferation from the transferred skin grafts.

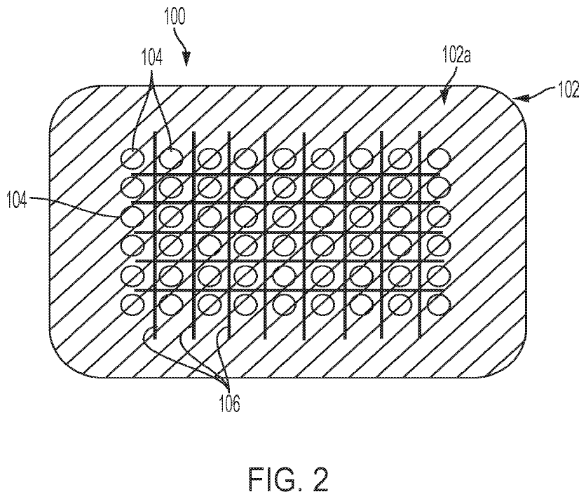

FIG. 2 schematically depicts another embodiment of a dressing 100 according to the present teachings, which includes a backing sheet 102 providing a plurality of adhesive sites 104 on a wound-facing surface 102a thereof. A plurality of strips 106 containing amniotic membrane material, e.g., amnion and/or chorion, are distributed across the surface 102a of the backing material in the form of a crisscross pattern. More specifically, in this embodiment, the plurality of strips 106 include two orthogonally intersecting sets of parallel strips, which are disposed between the adhesive sites 104.

Similar to the previous embodiment, the adhesive sites can be used to harvest a plurality of skin grafts, e.g., epidermal biopsies. Once the harvested skin grafts are transferred to a recipient site, the amniotic strips 106 can enhance the reepithelization of the recipient site. For example and without being limited to any particular theory, the amniotic strips can modulate the wound environment by supplying growth factors, anti-inflammatory cytokines and extracellular matrix (ECM) components, which can in turn enhance the process of reepithelization.

Similar to the previous embodiment, the backing sheet 102 can be formed of a suitable polymeric material. For example, the backing sheet 102 can be a breathable protective film such as polyurethane or can include an absorbent component such as foam. In some embodiments, the amniotic strips 106 can have a thickness, e.g., in a range of about 5 μm to about 80 μm and a width in a range of about 1 mm to about 10 mm.

One advantage of the dressing 100 is that it employs less amniotic material while still providing the advantages associated with the use of the amniotic material, e.g., enhanced re-epithelization of a wound site.

In some embodiments, the adhesive sites 104 can be formed by applying a suitable adhesive to the surface 102a of the backing sheet 102. As noted above, some examples of suitable adhesives can include, for example, silicone, acrylic, polyurethane, or hydrogen-based adhesives.

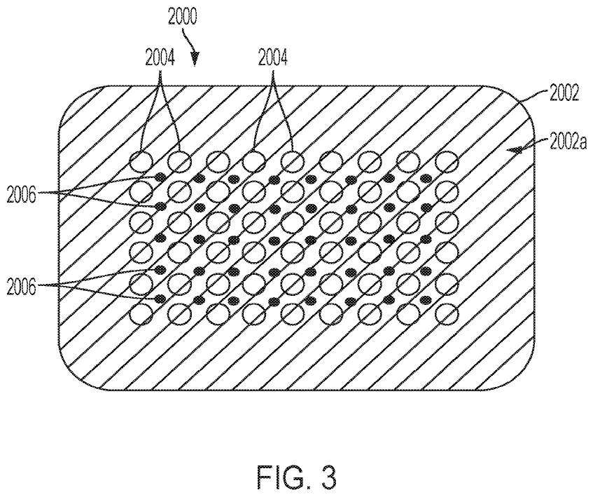

FIG. 3 schematically depicts a dressing 2000 according to another embodiment, which includes a backing sheet 2002 providing a plurality of adhesive sites 2004 distributed as a two-dimensional array across a wound-facing surface 2002a of the backing sheet 2002. In this embodiment, the adhesive sites 2004 have a substantially circular shape, though in other embodiments other shapes, e.g., polygonal shapes, can also be utilized. In this embodiment, the diameter of the substantially circular adhesive sites can be, for example, in a range of about 2 mm to about 10 mm. By way of example, the adhesive sites 2004 can be formed by coating selected portions of the wound-facing surface 2002a with a suitable adhesive, such as those discussed above. By way of example, the backing sheet 2002 can be formed of a suitable polymeric material, such as those discussed above. Further, a variety of adhesives, such as those discussed above, can be employed.

The dressing 2000 further includes a plurality of sites 2006 in the form of dots that contain amniotic membrane material, e.g., amnion and/or chorion. In this embodiment, the amniotic dots 2006 are distributed as a regular two-dimensional array among the adhesive sites 2004. In some embodiments, the dots can have a diameter, for example, in a range of about 1 mm to about 10 mm. The dots can be attached to the backing sheet via the adhesive coating of the backing sheet.

The adhesive sites 2004 can facilitate the capture of skin grafts from a donor site, and the amniotic material can modulate the wound environment by supplying growth factors, anti-inflammatory cytokines and extracellular matrix (ECM) components, which can in turn enhance the process of reepithelization.

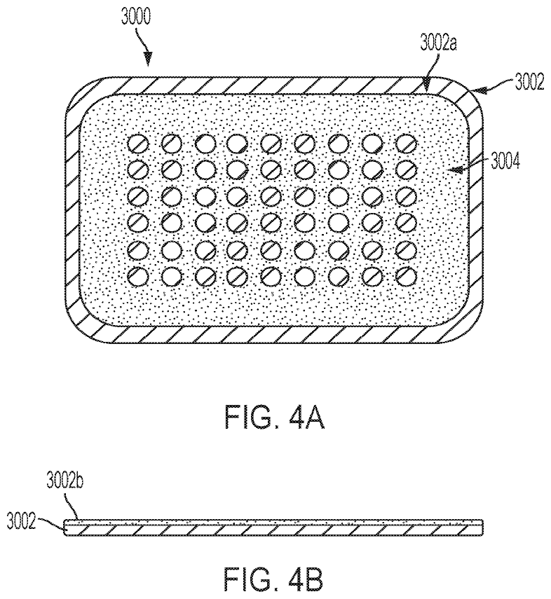

With reference to FIGS. 4A and 4B, in another embodiment, a dressing 3000 includes a backing sheet 3002 having a wound-facing surface 3002a to which an adhesive film 3002b is applied. A powder 3004 containing amniotic material, e.g., amnion and/or chorion, is applied to the adhesive film 3002b such that the powder coats most of the adhesive film 3002b other than a plurality of adhesive sites 3006. In this embodiment, the adhesive sites 3006 are substantially circular and arranged as a regular two-dimensional array. In other embodiments, the adhesive sites 3006 can have different shapes and can be distributed in a different way, e.g., randomly. In this embodiment, the adhesive sites 3006 can have a diameter, e.g., in a range of about 2 mm to about 10 mm, though other sizes can also be employed.

The backing sheet 3002 can be formed of any suitable polymeric material, such as those discussed above, and the adhesive can be, for example, any of the adhesives discussed above in connection with the previous embodiments.

In some embodiments, the powder 3004 can include a plurality of particles containing amniotic material (amnion and/or chorion), where the particles can have a maximum dimension, e.g., a diameter, in a range of about 1 micron to about 300 microns.

One advantage of the dressing 3000 is that it employs lower amounts of amniotic material while still providing the benefits of the amniotic material for facilitating the reepithelization of a wound site. In particular, similar to the previous embodiments, the adhesive sites 3006 can help capture skin grafts, e.g., epidermal grafts, and the powder containing amniotic material can modulate the wound environment by supplying growth factors, anti-inflammatory cytokines and extracellular matrix (ECM) components, which can in turn enhance the process of reepithelization.

The powder can provide a high surface area for the amniotic material, which can in some embodiments improve the speed of delivery of the relevant signaling proteins for enhancing the reepithelization of the wound site. Further, the sizes of the powder particles can be adjusted to change the surface area and hence modulate the residency time of the amniotic material applied to the wound site. In general, the larger particles will have a longer residency time relative to smaller particles.

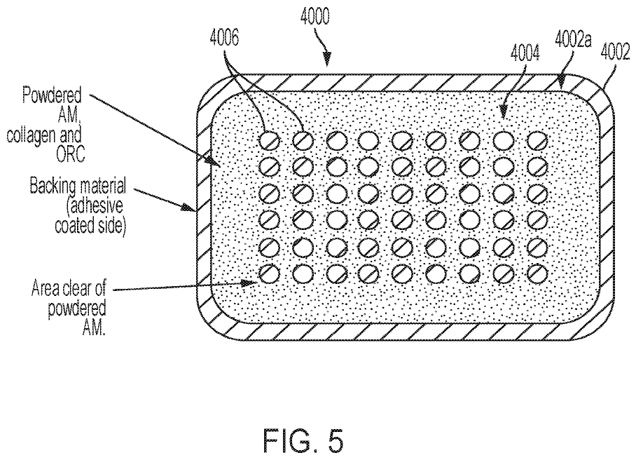

FIG. 5 schematically illustrates a dressing 4000 according to another embodiment, which includes a polymeric backing sheet 4002 having a wound-facing adhesive surface 4002a. By way of example, the adhesive surface 4002a can be in the form a coating of an adhesive, such as those discussed above.

A powder 4004 is applied to the adhesive surface 4002a such that a plurality of adhesive sites 4006 of the adhesive surface 4002a remain exposed while another portion of the surface is coated with the powder. In this embodiment, the powder includes a mixture of amniotic material, e.g., amnion and/or chorion, and collagen/ORC (oxidized regenerated cellulose). The adhesive sites 4006 can help capture skin grafts, e.g., epidermal skin grafts. The powder can in turn enhance the reepithelization of the wound site. In particular, as noted above, the amniotic material can modulate the wound environment by supplying growth factors, anti-inflammatory cytokines and extracellular matrix (ECM) components, which can in turn enhance the process of reepithelization. Further, in some embodiments, the collagen/ORC can provide protease modulation.

Another advantage of the use of the powder 4004 is that it imparts a high surface area to the amniotic material, which can improve the delivery speed of the relevant signaling proteins. Further, the sizes of the particles forming the powder and hence the surface area can be adjusted to modulate the residency time of the amniotic material applied to a wound site.

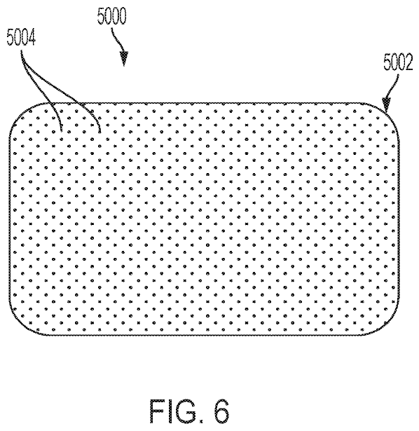

In some embodiments, a biopolymer film having adhesive properties is employed as the backing sheet in a dressing according to the present teachings and a powder containing amniotic material is dispersed on a wound-facing surface of the biopolymer film. By way of example, FIG. 6 schematically depicts such a dressing 5000, which includes a backing sheet 5002 that is formed of an adhesive biopolymer material. By way of example, the adhesive biopolymer film can be collagen/ORC based film.

In some embodiments, the biopolymer film can be a bioresorbable film composed of collagen/ORC combined with glycerol and/or glucose. By way of example, collagen can be present at 40% to 90% by weight or more preferably 50% to 60% by weight. The ORC component can be present at about 5% to about 45% by weight, or more preferably from about 35% to about 45% by weight. Addition of glucose can provide tack/adhesive properties; glucose can be present at 8% to 16% by weight. In some embodiments, glycerol can be included as a plasticizer to provide the film material with flexibility and can be added to in any suitable amount to impart a desired level of plasticity.

A powder 5004 containing an amniotic material, e.g., amnion and/or chorion, is applied to the wound-facing surface 5000a of the dressing. The adhesive property of the biopolymer material helps retain the powder particles on the wound-facing surface of the dressing. Further, the adhesive property of the biopolymer film facilitates the capture of one or more skin grafts. In other words, the wound-facing surface 5000a is tacky enough to allow the capture of skin grafts.

In some embodiments, the powder containing amniotic material can include a plurality of particles having a maximum dimension, e.g., a diameter, in a range of about 1 μm to about 300 μm. Similar to the previous embodiment, the amniotic powder can enhance the reepithelization of the wound site. In particular, as noted above, the amniotic material can modulate the wound environment by supplying growth factors, anti-inflammatory cytokines and extracellular matrix (ECM) components, which can in turn enhance the process of reepithelization.

In some implementations in which the biopolymer film forming the backing sheet 5002 is collagen/ORC based, the collagen/ORC can provide protease modulation, thereby enhancing the reepithelization of the wound site.

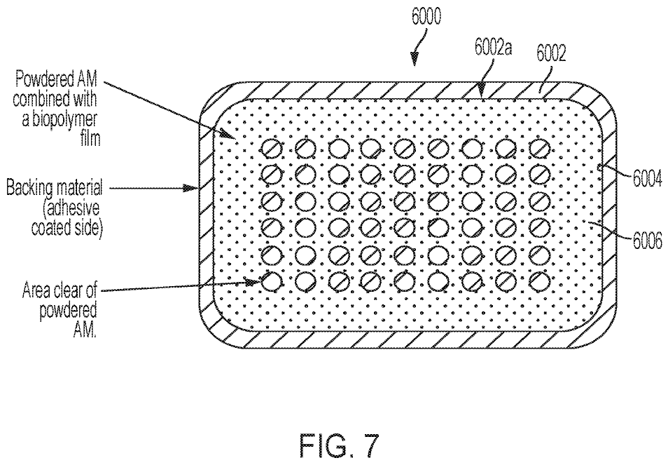

FIG. 7 schematically depicts a dressing 6000 according to an embodiment, which includes a backing sheet 6002 having an adhesive-coated surface 6002a. A biopolymer film 6004 having an amniotic material 6006, e.g., in the form of a powder, is coupled adhesively to the adhesive-coated surface 6002a of the backing sheet. In some embodiments, the biopolymer film can be tacky and the amniotic material 6006 can be applied to a tacky surface of the biopolymer film. Instead or in addition, the amniotic material 6006 can be incorporated within the biopolymer film. Similar to the previous embodiments, the backing sheet 6002 can be formed of any suitable polymeric material, such as those listed above. Further, the adhesive coating can be formed of any suitable adhesive, such as those discussed above. The biopolymer film can also be formed of any suitable material, such those discussed above, e.g., in connection with the dressing 10.

With continued reference to FIG. 7, in this embodiment, the biopolymer film 6004 includes a plurality of perforations 6005, which expose portions of the underlying adhesive surface 6002a, thereby forming a plurality of adhesive sites 6002b. Similar to the previous embodiments, the adhesive sites 6002b can help capture skin grafts. Further, the biopolymer film impregnated with the amniotic material can enhance the re-epithelization of a wound site when the dressing is used to transfer skin grafts to the wound site.

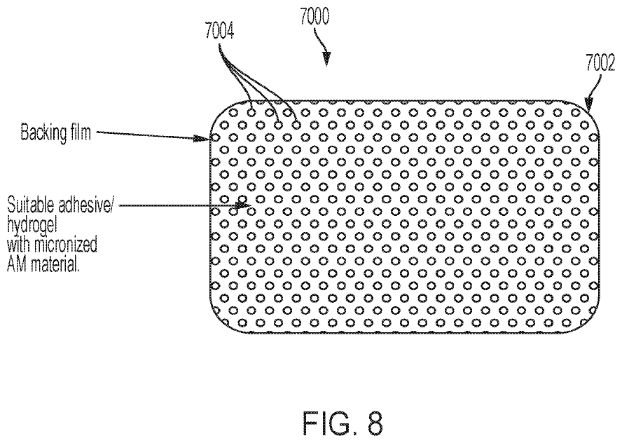

In another embodiment depicted schematically in FIG. 8, a dressing 7000 includes a backing film 7002 that is coated with a combination of powdered amniotic membrane material and a suitable adhesive or hydrogel 7004. In some such embodiments, the powdered amniotic membrane material (amnion and/or chorion) can be composed of a plurality of particles having a maximum dimension, e.g., a diameter, in a range of about 1 μm to about 300 μm. The adhesive can be, for example, any of those discussed above. Similar to a number of embodiments discussed above, the use of the powder allows lowering the amount of the amniotic material in the dressing while still providing the advantages associated with the use of the amniotic material, such as enhanced re-epithelization of a wound site. Further, the use of the powder of the amniotic material can advantageously enhance the surface area of the amniotic material effective for modulating the wound environment.

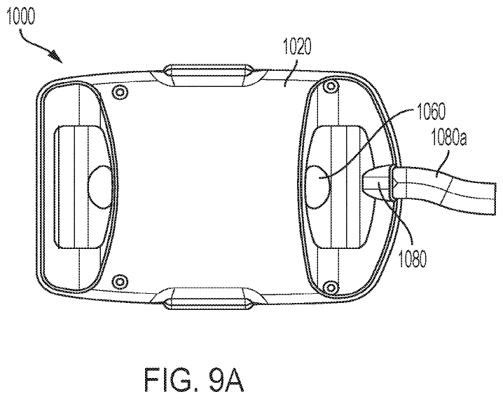

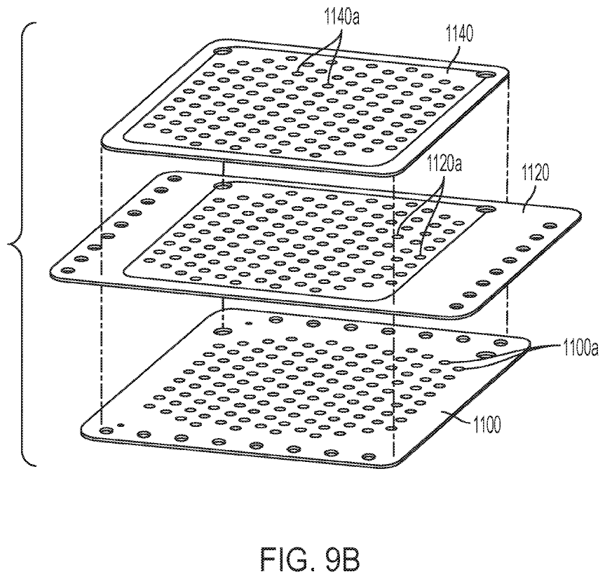

The dressings according to the present teachings can be employed to capture skin grafts generated by a variety of devices. By way of example, in some embodiments, the dressings can be employed to capture and transfer epidermal skin grafts generated by a device marketed by the assignee of the present application under the tradename Cellutome®. By way of example, a Cellutome® device can be employed to form such epidermal microblisters. For example, with reference to FIGS. 9A and 9B, such a device 1000 can include a head 1020 that can be removably and replaceably attached to a harvester (not visible in this figure) to form a hollow chamber 1060 into which a plurality of blisters can be drawn. In particular, the head 1020 can include a suction coupling 1080 that allows coupling the head via a suction tubing 1080a to a vacuum source (not shown) to generate a negative pressure within the chamber. The harvester includes an orifice plate 1100 having a plurality of openings 1100a through which a plurality of epidermal microblisters can be raised into the hollow chamber 1060. The harvester further includes a cutter plate 1120 disposed between the orifice plate 1100 and an upper plate 1140. Both the cutter plate 1120 and the upper plate 1140 include a plurality of openings 1120a and 1140a, respectively, through which the blisters can protrude. Once the blisters are formed, the cutter plate 1120 can be moved relative to the orifice plate and the upper plate to cut the blisters. Further details regarding such a blister-generating device can be found, e.g., in U.S. Pat. No. 9,173,674 titled “Devices for Harvesting a Skin Graft,” which is herein incorporated by reference in its entirety.

More specifically, once the blisters are formed, the application of a reduced pressure to the chamber 1060 can be discontinued, and the head portion 1020 can be removed to expose the epidermal blisters. A dressing according to the present teachings can then be placed over the epidermal blisters (step 2 in the flow chart of FIG. 7). In some embodiments, the number of the capture sites provided on the dressing is equal to the number of the formed epidermal blisters and the distribution of the capture sites across the skin-graft receiving surface of the dressing is such that there is a one-to-one correspondence between the capture sites and the epidermal blisters.

The epidermal blisters can then be cut and the cut blisters can be captured by the skin-graft capture sites of the dressing (step 3 in the flow chart of FIG. 7). The dressing can then be placed on a recipient site, e.g., a wound site, to transfer the harvested blisters to a recipient site, e.g., a wound, so as to promote re-epithelization of the recipient site.

Those having ordinary skill in the art will appreciate that various changes can be made to the above embodiments without departing from the scope of the invention.

1. A dressing for transferring skin grafts from a donor site to a recipient site, the dressing comprising:
a perforated amniotic membrane sheet having a skin-graft contacting surface and an opposed surface; and
a backing sheet having a surface coupled to said opposed surface of the amniotic membrane sheet.

2. The dressing of claim 1, wherein said perforated amniotic membrane sheet comprises one or more perforations configured for exposing a plurality of portions of the backing sheet for harvesting one or more epidermal skin grafts.

3. The dressing of claim 2, wherein said one or more perforations comprise a width in a range of about 2 mm to about 10 mm and a thickness in a range of about 5 μm to about 80 μm.

4. (canceled)

5. The dressing of claim 2, wherein said surface of the backing sheet is coated at least partially with an adhesive and configured to provide one or more adhesive sites in substantial register with said one or more perforations in order to facilitate harvesting of epidermal skin grafts via said adhesive sites.

6. The dressing of claim 1, wherein said backing sheet comprises a polymer, the polymer comprising at least one of: a polyurethane, a polyolefin, a cyclic olefin copolymer, a polyamide, a polyester, a polyether, a co-polyester and any of a copolymer or a blend thereof.

7. The dressing of claim 2, wherein said perforations are distributed across said amniotic sheet as a regular array.

8. The dressing of claim 1, wherein said amniotic sheet comprises at least one of: amnion and amnion and chorion.

9. (canceled)

10. (canceled)

11. The dressing of claim 1, wherein said backing sheet has a thickness in a range of about 15 μm to about 100 μm.

12. The dressing of claim 1, wherein said perforated amniotic membrane is a dehydrated membrane.

13. The dressing of claim 1, wherein the perforated amniotic membrane sheet comprises one or more amniotic membrane sites distributed across the surface of the backing sheet and configured such that the one or more adhesive sites remain exposed for harvesting the one or more epidermal skin grafts.

14. The dressing of claim 13, wherein said amniotic membrane sites comprise one or more strips including amniotic material.

15. The dressing of claim 13, wherein said amniotic membrane sites comprise a plurality of strips distributed across said surface of the backing sheet so as to form a crisscross pattern, wherein said plurality of strips include an amniotic material.

16. The dressing of claim 15, wherein said one or more adhesive sites comprise a plurality of adhesive sites distributed across said surface such that any two adhesive sites are separated by at least a portion of one of said strips.

17. The dressing of claim 16, wherein said adhesive sites are substantially circular.

18. The dressing of claim 13, wherein said one or more amniotic portions comprise a plurality of dots distributed across said surface of the backing sheet.

19-25. (canceled)

26. A dressing for transferring skin grafts from a donor site to a recipient site, the dressing comprising:
a backing sheet having a surface comprising one or more adhesive sites; and
a powder comprising an amniotic membrane material, said powder being distributed across at least a portion of said surface of the backing sheet such that said one or more adhesive sites remain exposed for harvesting one or more skin grafts.

27. The dressing of claim 26, wherein said powder comprises a plurality of particles having a size in a range of about 1 to about 300 microns.

28. The dressing of claim 26, wherein said one or more adhesive sites are distributed as a regular array across said surface of the backing sheet.

29. The dressing of claim 26, wherein said one or more adhesive sites are substantially circular.

30. The dressing of claim 26, wherein said powder further comprises at least one of collagen and oxidized regenerated cellulose (ORC).

31. (canceled)

32. A dressing for transferring skin grafts from a donor site to a recipient site, the dressing comprising:
a biopolymer film having a surface adapted for harvesting skin grafts; and
a powder comprising an amniotic membrane material, said powder distributed across at least a portion of said surface of said adhesive biopolymer film.

33. The dressing of claim 32, wherein said biopolymer film comprises at least one of collagen and oxidized regenerated cellulose (ORC).

34. (canceled)

35. The dressing of claim 32, wherein said powder comprises at least one of amnion and chorion.

36-37. (canceled)

38. The dressing of claim 32, further comprising a backing sheet having an adhesive surface, and wherein the biopolymer film comprises a surface coupled to said adhesive surface of the backing sheet and an opposed surface, and the powder is distributed over said opposed surface of the biopolymer film.
